(19) United States
(12) Patent Application Publication
(10) Pub. No.: US 2015/0352280 A1
(43) Pub. Date: Dec. 10, 2015

(54) INSULIN PUMP APPARATUS, DEVICE, METHODOLOGY AND KIT

(71) Applicant: Timothy Allen Deratany, Miramar, FL (US)

(72) Inventor: Timothy Allen Deratany, Miramar, FL (US)

(21) Appl. No.: 14/728,969

(22) Filed: Jun. 2, 2015

Related U.S. Application Data
(60) Provisional application No. 62/007,831, filed on Jun. 4, 2014.

(51) Int. Cl. A61M 5/168; A61M 5/172; A61M 5/00; A61B 5/145; A61M 5/142; A61M 31/00

(52) U.S. Cl. CPC A61M 5/16877; A61M 5/142; A61M 5/168; A61M 31/00; A61M 5/003; A61B 5/14532; A61M 5/1723; A61M 2005/14208; A61M 2205/18; A61M 2205/50

(57) ABSTRACT

A smart insulin pump that is programmable to customize for the physical and mental conditions of a user. A user may input information that may be analyzed by the insulin pump to deliver insulin with appropriate bolus type and basal rate, such as particular or customizable to that user or a group. The insulin pump, apparatus, system, technique, methodology and kit of the present invention includes a computerized controller to analyze, and input and control operations of the pump, delivering the bolus of insulin at an adaptable and varying basal rate better suited to the characteristics of the patient in question.

### CROSS REFERENCE TO PRIORITY APPLICATION

The present invention claims priority to U.S. Provisional Patent Application No. 62/007,831, filed Jun. 4, 2014, the contents of which are incorporated herein in their entirety by reference.

### BACKGROUND OF INVENTION

The importance of medicine and health care to modern society is immeasurable. New treatments, procedures and medicaments have transformed the world. With the rise of obesity and diabetes in the United States and the world, the treatment and amelioration of the damaging consequences of diabetes are major health initiatives, affecting many tens of millions.

Current treatments for diabetes, however, are mired in old paradigms and equipment that follow conventional paradigms of thought regarding insulin usage, despite the research and developments of recent years demonstrating the need for better tracking and treatment of fluctuating or variable insulin levels. In particular, prior paradigms focus on the constant delivery of insulin to maintain stable blood glucose.

There is, therefore, a present need to provide an improved technique and apparatus to deliver insulin to patients, better monitor the dynamic status of insulin usage for the patient, monitor and variably address imbalances in the metabolism of the patient that affect insulin delivery, as well as address the differing means and timing of insulin introduction.

The present invention is directed to making these improved techniques, apparatuses and kits simple to use, inexpensive, accurate and adaptable for use by all diabetics.

### SUMMARY OF THE PRESENT INVENTION

The invention generally relates to a smart insulin pump that is programmable to customize for the physical and mental conditions of a user. A user may input information that may be analyzed by the insulin pump to deliver insulin with appropriate bolus type and basal rate, such as particular or customizable to that user or a group. The insulin pump, apparatus, system, technique, methodology and kit of the present invention includes a computerized controller to analyze, and input and control operations of the pump, delivering the bolus of insulin at an adaptable and varying basal rate better suited to the characteristics of the patient in question.

### BRIEF DESCRIPTION OF THE DRAWINGS

While the specification concludes with claims particularly pointing out and distinctly claiming the subject matter that is regarded as forming the present invention, it is believed that the invention will be better understood from the following description taken in conjunction with the accompanying DRAWINGS, where like reference numerals designate like structural and other elements, in which:

FIG. 1 is a representative view of an embodiment of a device incorporating the principles of the present invention in an operative mode;

FIG. 2 is a representative view of a case or kit that includes the device of FIG. 1 therein, along with accessories and component parts thereof;

FIG. 3 is an illustration of a triangular-shaped configuration representative of insulin delivery over a period of time pursuant to a protocol embodiment of the present invention;

FIG. 4 is an illustration of a diamond-shaped configuration representative of insulin delivery over a period of time pursuant to a second protocol embodiment of the present invention;

FIG. 5 is an illustration of an irregular-shaped configuration representative of insulin delivery over a period of time pursuant to a another protocol embodiment of the present invention;

FIG. 6 is a flowchart illustrating aspects of the present invention involving insulin monitoring and adjustable usage pursuant to the teachings of the present invention; and

FIG. 7 is another flowchart illustrating aspects of the present invention, as also shown in FIG. 6, involving adjustable insulin delivery or dispersal according to the present invention.

### DETAILED DESCRIPTION OF THE PRESENT INVENTION

The present invention will now be described more fully hereinafter with reference to the accompanying DRAWINGS, in which preferred embodiments of the invention are shown. It is, of course, understood that this invention may, however, be embodied in many different forms and should not be construed as limited to the embodiments set forth herein; rather, these embodiments are provided so that the disclosure will be thorough and complete, and will fully convey the scope of the invention to those skilled in the art. It is, therefore, to be understood that other embodiments can be utilized and structural changes can be made without departing from the scope of the present invention.

The present invention is generally directed to improved apparatuses, systems, processes and techniques for usage by individuals in their daily handling for diabetes, and in hospitals, medical and other health care facilities for same.

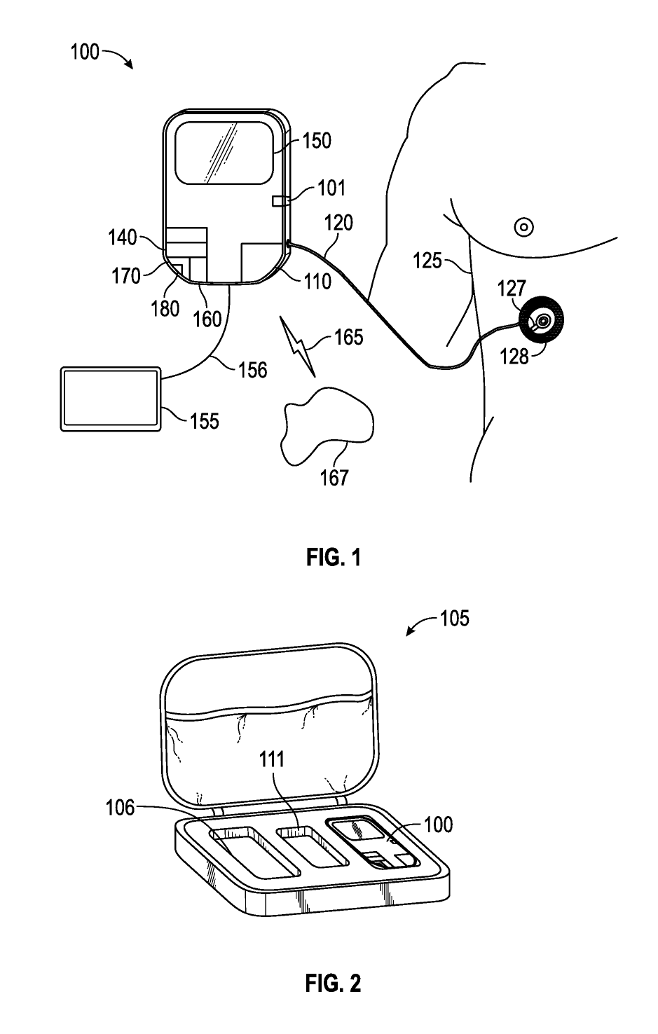

With reference now to FIG. 1 of the DRAWINGS, there is illustrated a representative configuration of a smart insulin pump pursuant to the present invention, generally designated by the reference numeral 100. As illustrated, the pump 100 includes a container or store, generally designated by the reference numeral 110, within which insulin is contained until administration, as is known in the art. It should, of course, be understood that the container 110 may be a cartridge, vial or other such fungible container for insulin that may be inserted and swapped out into the pump 100, where used cartridges are then disposed.

As also shown in FIG. 1, attached to the container 110 is a tube or hose, generally designated by the reference numeral 120, which when operational connects to a patient, generally designated by the reference numeral 125, providing a point of administration for the insulin, such as along the abdomen. It should be understood that the tube 120 when not in use may be stored within a compartment of the pump 100 or in a case, which may house the pump 100 and the various components in a kit form, as shown and described in more detail hereinbelow in connection with FIG. 2.

As is understood in the art, a cannula or other insertion device, generally designated by the reference numeral 127, may be employed to insert and administer the insulin to the patient or user 125 at an insertion area, generally designated by the reference numeral 128, on the stomach or other area of the user 125, as is understood in the art.

As discussed, the cannula 127, along with other diabetic components and paraphernalia can be stored within a case or kit 105, described further hereinbelow. It should also be understood that the insulin (or other medicament) may alternatively be administered orally, pursuant to the regimen and other protocols set forth hereinbelow in connection with alternative embodiments of the present invention.

As also shown in FIG. 1, the pump 100 includes a processor, generally designated by the reference numeral 130, a memory, generally designated by the reference numeral 140 and an interface, generally designated by the reference numeral 150. As is understood in the art, the interface 150 may comprise a display, buttons, virtual or actual, and other interaction means to convey information and data regarding the insulin levels and other data to the user.

As discussed, the present invention generally relates to a smart insulin pump 100 that is programmable to customize physical and mental conditions of the user with programs, which may be stored within the aforesaid memory 140. A user may, for example, input information, such as via the interface 150, which may be analyzed by the insulin pump 100, via the processor 130, to deliver insulin (or instruct the user therefor) with appropriate bolus type and basal rate, such as via the tube 120 and cannula 127, as discussed. In a preferred embodiment of the present invention, the processor or computerized controller 130 analyzes all input and control operations of the pump 100.

The smart insulin pump 100 preferably includes bolus or dosage tracking, which allows the user to see in real time how the insulin used in the bolus is acting, such as on the interface 150, or on a monitor or the like connected to the pump 100, such as generally designated by the reference numeral 155, by a wireline or wireless connection, generally designated by the reference numeral 156. It should also be understood that the monitor 155 could constitute a computer, a computer display, a smart phone screen, an iPad or other tablet display, or other consumer, hospital or industrial displays, as are understood in the art. Bolus tracking may be performed by various feedback graphs. The smart insulin pump 100 preferably also follows the action of the insulin it uses, which may be found in clinical research or patient specific observations, i.e., the pump 100 anticipates the insulin usage based on the characteristics and data known about the patient, group or other sampling.

Additionally, a glycemic index library, generally designated by the reference numeral 160, is preferably used to coordinate consuming a type of food or drink before or after onset of a bolus, such as through the aforesaid interface 150. It should be understood that all or portions of the aforesaid glycemic index library 160 may reside within the memory 140. However, it should be understood that some or all of these values may alternatively reside externally to the pump 100 and memory 140, such as with the aforesaid computer 155, or with the glycemic data being made available, e.g., via wireline or wireless connection, generally designated by the reference numeral 165, to the Internet, generally designated by the reference numeral 167, as is understood in the art, with perhaps only pertinent, user-selected or often-used glycemic data being resident within memory 140. The smart insulin pump 100 of the present invention preferably combines these advanced timing metrics with intelligent insulin delivery to provide better pump function, and, therefore, better diabetes care, contouring the insulin delivery in quantity and over time.

The smart insulin pump 100, kit and methodology of the present invention preferably include a timing component to alert the user of an optimal time to eat, administer a bolus, or perform an activity. A timing component of this type, generally designated by the reference numeral 170, which may also reside in said memory 140, is preferably customizable to the aforementioned physical and mental conditions of the user, utilize the aforesaid glycemic index 160, and/or offer high or low blood glucose (“BG”) correction solutions, as calculated by said processor 130 from an algorithm or technique for same, such as may be stored in said memory 140.

The timing component 170 preferably additionally includes a timing feature, which alerts a user when a bolus is active enough to consume a meal or vice versa. It should be understood that the aforementioned timing feature, generally designated by the reference numeral 180, may be visual, vibratory and/or auditory to alert the user, i.e., the pump 100 may include these and other sensory alerts, generally designated by the reference numeral 101.

A basal prompt may be included to control an insulin level deliverable to a user, such as via the interface 150. A lower basal setting may be selected to deliver less insulin. A normal activity basal setting may be selected to deliver normal insulin levels. Additionally, a high basal rate may be selected to deliver increased insulin levels, such as for low activity, sick days, high levels of stress, and other factors, with all of these respective values stored in the memory 140 and/or externally, as described. The basal prompt may prompt the wearer, such as patient 125 of the pump 100, to adjust the basal rate at preset or varying intervals for a settable number of hours. If no basal rate is selected by the user, the pump 100 preferably defaults to normal activity basal settings, which may, of course, be a customized normal setting based on an individual or a group.

The bolus may be adjusted by decreased or accelerated user selection. For example, in situations anticipating high-fat and protein food with slow metabolizing affects or gastroparesis, a so-called triangle wave bolus delivery configuration may be used.

As discussed and with reference now to FIG. 2 of the DRAWINGS, the aforementioned pump 100 and all of the accessories therefor may be included in a case or a kit, generally designated by the reference numeral 105, for ease of patient or hospital use, similar to the kits already employed for diabetics' usage but incorporating the improvements set forth in the present invention. The case 105 may include additional insulin, such as in a bottle or other container, generally designated by the reference numeral 111, and a compartment 106 within which to store the various paraphernalia and components for use with the pump 100, such as the tube 120, cannula 127 and connector 156, as well as power sources, such as batteries, that power the pump 100 and/or recharging connectors thereto, as is understood in the art.

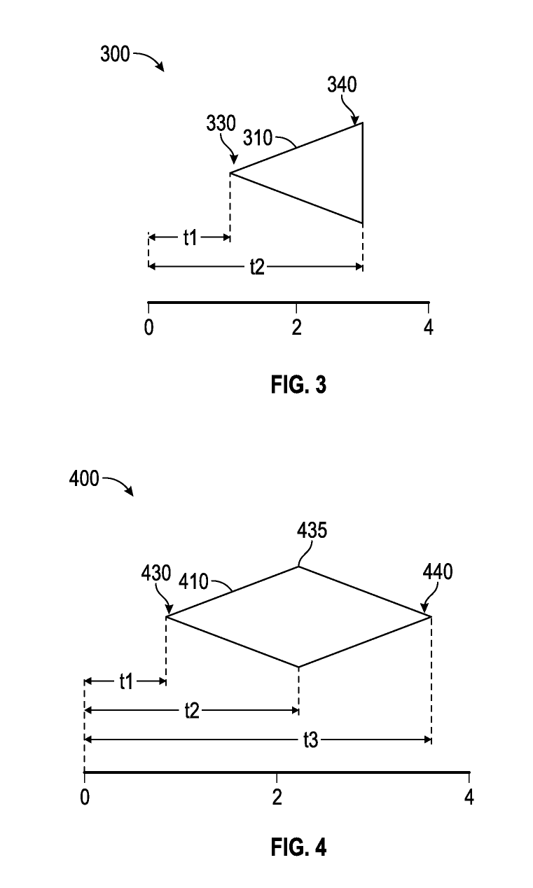

With reference now to FIG. 3 of the DRAWINGS, there is shown a first embodiment of an insulin distribution technique or protocol pursuant to the teachings of the present invention, generally designated by the reference numeral 300, employing the aforementioned pump 100 and the features. In this first embodiment, the insulin is distributed pursuant to the aforementioned triangular pattern, generally designated by the reference numeral 310, over a period selectable by user, here measured in hours. In this configuration, upon pump 100 initiation, at time period zero or 0, insulin delivery is delayed by a period t1, at which point insulin delivery commences in the aforesaid triangular configuration delivery, i.e., the dosage or basal rate starts small and increases. It should, of course, be understood that t1 can be zero as well, in which insulin delivery is immediately initiated.

The front end of the triangle or the left most portion thereof in FIG. 3, generally designated by the reference numeral 330. After period t1, the small area of the vertical cross section of the triangle 310 is representative of the amount of the dosage, where a relatively small amount of insulin is dispensed initially that increases as time progresses and the representative triangle area increases, resulting in the majority of the bolus being delivered towards the end of the triangular interval, generally designated by the reference numeral 340.

Thus, upon activation of the device or pump 100, employing the triangular basal rate delivery technique or protocol 200, insulin delivery is delayed, then, at time t1, the insulin is gradually introduced, increasing arithmetically or linearly in this example, for a determined period of time, and then terminated at time point t2. In this fashion, basal rate of the bolus delivered varies moment by moment, dependent upon the program, algorithm or instructions determined by the processor 130 for that patient 125. As discussed, the user could also initiate the aforementioned triangular (or other) insulin delivery mechanism, such as by inputs to the interface 150.

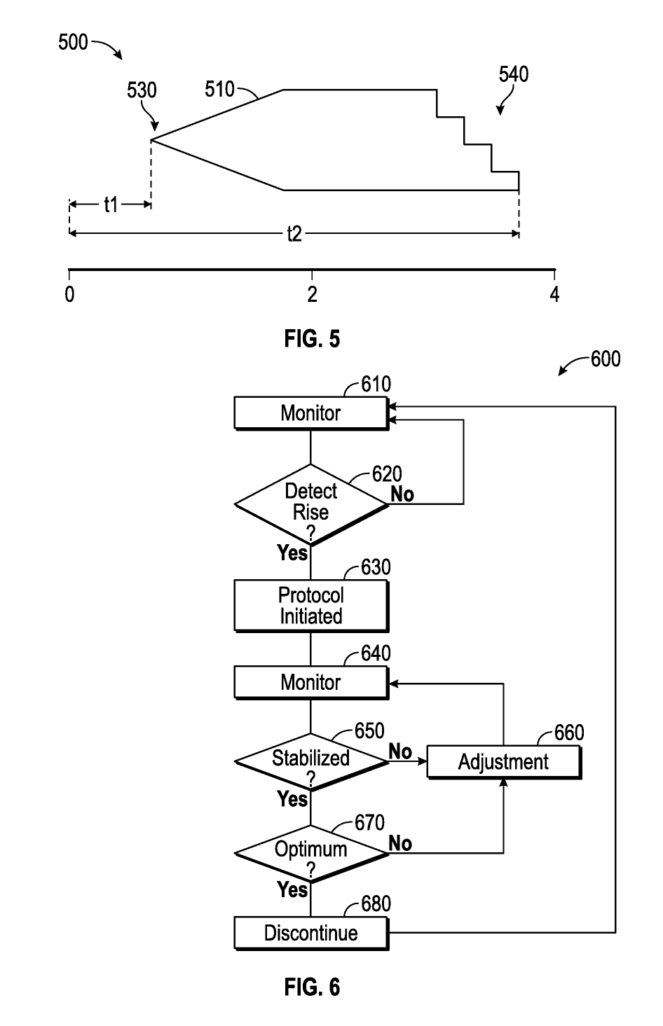

Alternatively, with reference now to FIG. 4 of the DRAWINGS, there is shown a second embodiment of an insulin distribution or delivery technique, protocol or paradigm pursuant to the teachings of the present invention, generally designated by the reference numeral 400. In this second embodiment, the insulin is alternatively distributed pursuant to a diamond wave bolus, which is used to dispense the bolus in a diamond-shaped pattern, generally designated by the reference numeral 410, over time. As with the aforedescribed triangular configuration illustrated and described in connection with FIG. 3, the front end of the diamond 410, generally designated by the reference numeral 430, dispenses a relatively small amount of insulin starting after a delay time period t1, the amount then increasing with time to a center time period t2 of maximal dispensing, generally designated by the reference numeral 435, then gradually decreasing to deliver less insulin to where the insulin administration period concludes, generally designated by the reference numeral 440, at time period t3. It should be understood that although the pre-maximal and post-maximal dispensing amounts and time are the same in this embodiment, the contours of the actual patient insulin delivery may vary both in amount and in time, meaning that the shape will alter, as illustrated and described in more detail in connection with FIG. 5.

It should, of course, be understood that in both of the aforesaid embodiments, the time of onset of insulin administration, i.e., time period t1, may be delayed by a customizable amount. Further, the amount of insulin delivered can be controlled by the pump 100 to accelerate or decelerate the form of the bolus, whether arithmetically, linearly or some other customizable configuration, which is a great departure from the simplified and static approaches of the prior art techniques with constant basal rate deliveries of insulin.

With reference now to FIG. 5 of the DRAWINGS, there is shown another embodiment of an insulin distribution or delivery technique, protocol or paradigm pursuant to the teachings of the present invention, generally designated by the reference numeral 500. It should be understood that the insulin delivery contour or configuration may be of almost any shape. For example, in this embodiment of the present invention, the shape is an irregular configuration, generally designated by the reference numeral 510, which can be calculated by the processor 130 or input by the user from memory 140 or from a database, such as information stored remotely across the Internet 167.

In this embodiment, after time period t1, insulin starts being administered, as represented by the front end of the shape 510, generally designated by the reference numeral 530, and then levels off for some time, before a staggered decrease at the end portion of the configuration 510, generally designated by the reference numeral 540, discussed further hereinbelow.

Employing the pump 100 components, the user or patient 125 is able to govern or control additional facets of their (or another's) insulin usage dynamically. For example, after insulin injection or insertion, employing the aforesaid triangular, diamond, irregular or other representative insertion protocols, both Active and Future Active insulin may be tracked and monitored by the user 125, such as via the interface 150, display 155 or other visual (or for the deaf auditory) monitors or alerts 101, as described.

It should be understood that Active insulin is insulin currently or immediately in use or active in the patient, where this number of amount or estimate is based upon the manufacturer's research of the administered insulin's peak times over a population or over a group similar to the statistics or characteristics of the user 125, perhaps even personalized over time with the user's prior usages. In any event, the amount of the aforesaid Active insulin is monitored.

Similarly, Future Active insulin is insulin that will become active in the future, whereas the Active insulin is presently or directly active in the body, as described. Based upon the aforementioned manufacturer's research, The Future Active type of insulin may also be monitored after administration and the effects and characteristics monitored by the user 125, on the interface 150, display 155 or otherwise, alone or alongside the Active insulin monitoring and other information.

With reference now to FIG. 6 of the DRAWINGS, there is shown a flowchart, generally designated by the reference numeral 600, illustrating some basic functionality of the pump 100 pursuant to the teachings of the present invention, regarding the measurement and monitoring of the active insulin injected or administered into a patient or user, as well as the dynamic adjustments to the insulin delivery mechanism.

As shown in FIG. 6, the blood glucose level of a patient is monitored, as generally designated by the reference numeral 610. It should be understood that in a preferred embodiment this monitoring is dynamic, such as done via continuous glucose monitoring (CGM) measurements, as is understood in the art. In any event, periodically, the BG level is measured to detect if there has been a change in the blood glucose level, generally designated by the reference numeral 620. If no change, then monitoring and periodic testing continues, as described. If, however, there has been a BG change, which usually constitutes an increase, then the protocol of the present invention is initiated, generally designated by the reference numeral 630, such as the protocols illustrated and described in connection with FIGS. 3-5.

With the protocol 630 in place, the pump 100 then monitors the progress of the protocol administration, generally designated by the reference numeral 640. As above, the system periodically measures the blood glucose level to determine if the BG level has stabilized, generally designated by the reference numeral 650. If the levels have not stabilized, then an adjustment is needed, generally designated by the reference numeral 660, described in more detail hereinbelow. For example, the protocol 630 may require a particular degree of blood glucose improvement in response to the protocol treatment in question, such as the triangular protocol of FIG. 3, or various phases in the other protocols. Where the blood glucose levels are off, i.e., not in line with the expected, calculated amounts and therefore no stabilized 650, then adjustment 660 may be required.

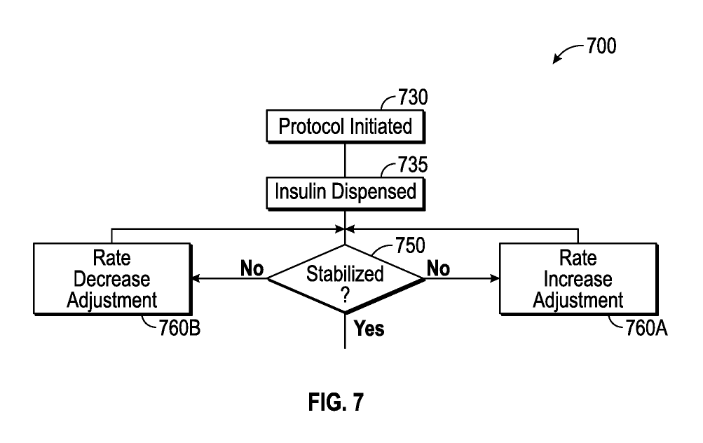

It should be understood that the adjustment 660 in the pump 100 uses the patient's blood glucose number, more particularly a BG number that incorporates that patient's correction factor, to determine the bolus thusfar and whether a bolus increase, decrease or no action is required. It should be understood that the adjustment 660 is preferably based on the real time or dynamic blood glucose level readings. In any event, after the adjustment 660 is made, the device and technique of the present invention returns to monitoring 640 and detecting 650 for measurement out of the expected range. Further details about the adjustment 660 functionality are illustrated and described in hereinbelow in connection with FIG. 7.

With further reference to FIG. 6, when the blood glucose levels for the initiated protocol 600 have stabilized 650, then a determination is made whether an optimum level has been reached, generally designated by the reference numeral 670. If not, then determinations are made for further adjustments 660, as described. If, however, optimum targets have been attained, i.e., the patient's blood glucose levels are normal, then the insulin delivery is terminated, generally designated by the reference numeral 680, and the protocol 600 and the pump 100 returns to the normal blood glucose monitoring 610 as described.

As shown in FIGS. 3-5, the particular configuration shown operate over particular periods of time. After adjustment 660, however, those time periods, as well as the amounts of the dosage, i.e., the representative shape of the configuration, may change to better accommodate the dynamic conditions and needs for the patient or user, and new insulin levels and periods calculated and employed thereby. With further adjustment 660, event those new configurations may be changed further, and so on, until optimum 670 is reached and the insulin delivery terminated 680.

With reference now to FIG. 7 of the DRAWINGS, there is illustrated a flow chart modified from that shown and described in connection with FIG. 6 hereinabove, generally designated by the reference numeral 700. As shown, after protocol initiation, generally designated by the reference numeral 730, insulin delivery or dispersal is begun, generally designated by the reference numeral 735. As discussed, periodically the dynamic, real time blood glucose levels are ascertained to determine if the BG level has stabilized, generally designated by the reference numeral 750. If not stabilized 750, e.g., the blood glucose levels remain high, then the amount and duration of the insulin may need to be increased, generally designated by the reference numeral 660A, as described in detail hereinabove.

If, however, the non-stabilized 750 blood glucose level is due to lower than expected BG levels, then there may be a determination to decrease the amount and/or duration of insulin, generally designated by the reference numeral 660B. IN both instances, the blood glucose levels are re-measured and the protocol 700 reassessed, i.e., whether stabilization 750 has been attained. It should be understood that multiple such adjustments can be made, e.g., the stepped nature of the insulin reduction 540 in the irregular configuration 510 of FIG. 5 is representative of an adjustable protocol made in real time. Of course, once stabilization 750 has been attained, then insulin delivery can terminate, as discussed hereinabove.

By way of example, the technique or algorithm of the present invention, which may be stored in memory 140 or externally as described, attempts to keep the blood glucose levels of the patient or user within a target range, i.e., stabilized and optimized. As discussed, these calculations, of course, accommodate the particular user's correction factor or insulin sensitivity, an amount determinable for each individual and capable of estimate. Thus, the desired target range is adjusted accordingly.

With each dosage of insulin administered, the device 100 computes from the aforementioned BG, such as via CBG or a BG reading packet, an action to take. For example, for each mg/dl above the desired blood glucose target, the following amount is determined:

mg/dl above target divided by patient corrective factor,

and that amount of insulin is immediately dosed after the blood glucose reading packet is processed.

If the next blood glucose packet or level measurement shows a drop in blood glucose above the range, then no further insulin is dosed, and if the next blood glucose packet shows no change, then the following amount is determined:

mg/dl above target divided by (patient corrective factor divided by 2),

and that amount of insulin is immediately dosed after the blood glucose reading packet is processed.

If, however, the next blood glucose packet or level measurement shows no change or a small increase (10 mg/dl or less) in blood glucose over the range, then the following amount is determined:

mg/dl above target divided by (patient corrective factor divided by 2),

and that amount of insulin is immediately dosed after the blood glucose reading packet is processed.

Finally, if the next blood glucose packet or level measurement shows a rise in blood glucose over 10 mg/dl, then the following amount is determined:

mg/dl above target divided by patient corrective factor,

and that amount of insulin is immediately dosed after the blood glucose reading packet is processed. It should be understood that the aforementioned actions are illustrative of the various actions that may be determined pursuant to the teachings and principles of the present invention.

It should be understood that on initial setup and continual usage, the pump 100 may ask if the patient tends to run on a low or high blood glucose or BG side by percent and/or BG figure. The pump 100 may also ask at which time of day the patient is running lower or higher BG and suggest the patient's insulin be adjusted for basal rate and/or bolus during those periods of fluctuation. The pump 100 may also use a CGM device for some time preceding use of the smart insulin pump to gather injection related information that may optimize use of bringing BG levels to normal before food consumption or activity.

Additionally, the smart insulin pump 100 may include a setup feature for configuring basal rate, carbs to bolus ratio, correction factor, and other operational parameters of the pump. The setup feature may customize delivery of insulin using the processor or controller 130 and reducing the need for a clinical determination. Factors may be based using the “1800 rule,” the “1500 rule,” physical, and/or mental parameters, as is understood to those of skill in the art. Physical parameters include weight, height, gender, activity level, and other contributing health conditions. Mental parameters include stress, anxiety, depression, medication, and other contributing mental health conditions. All of these parameters and data may be entered using the aforesaid interface 150 or otherwise downloaded into memory 140, as is understood in the art. The techniques or algorithms of the present invention ascertain and implement an insulin protocol for the particular user or patient with these statistics or health data.

The previous descriptions are of preferred embodiments for implementing the invention, and the scope of the invention should not necessarily be limited by these descriptions. It should be understood that all articles, references and citations recited herein are expressly incorporated by reference in their entirety. The scope of the current invention is defined by the following claims.

What is claimed is:

1. An insulin pump comprising:
a controller, said controller controlling the administration of insulin to a user; and
an insulin delivery mechanism connected to said controller, said insulin delivery mechanism capable of delivering insulin to said user,
wherein said controller dynamically monitors bolus and basal rate levels of the delivery of said insulin to said user, and adjusts said insulin delivery mechanism to dynamically decrease or increase the delivery of said insulin to said user.

2. The insulin pump according to claim 1, wherein the bolus level of insulin delivery to said user is customizable to said user and an activity of said user.

3. The insulin pump according to claim 1, wherein the bolus level of insulin delivery to said user is customizable by said user.

4. The insulin pump according to claim 1, wherein the basal rate level of insulin delivery to said user is customizable to said user and an activity of said user.

5. The insulin pump according to claim 1, wherein the basal rate level of insulin delivery to said user is customizable by said user.

6. The insulin pump according to claim 1, wherein the insulin is administered orally.

7. The insulin pump according to claim 1, further comprising:
a timing component, said timing component capable of alerting said user to perform an activity, said activity comprising eating.

8. The insulin pump according to claim 1, wherein the bolus and basal rate levels of the delivery of said insulin to said user conform to a triangular pattern of insulin delivery.

9. The insulin pump according to claim 1, wherein the bolus and basal rate levels of the delivery of said insulin to said user conform to a diamond pattern of insulin delivery.

10. The insulin pump according to claim 1, wherein the bolus and basal rate levels of the delivery of said insulin to said user conform to an irregular pattern of insulin delivery.

11. A kit for diabetics comprising:
an insulin pump with a controller, said controller controlling the administration of insulin to a user;
an insulin delivery mechanism connected to said controller and to said user, said insulin delivery mechanism capable of delivering insulin to said user,
wherein said controller dynamically monitors bolus and basal rate levels of the delivery of said insulin to said user, and adjusts said insulin delivery mechanism to dynamically decrease or increase the delivery of said insulin to said user through said insulin delivery mechanism.

12. The kit according to claim 11, wherein the bolus and basal rate levels of insulin delivery to said user are customizable to said user and an activity of said user.

13. The kit according to claim 11, wherein the bolus and basal rate levels of insulin delivery to said user are customizable by said user.

14. The kit according to claim 11, wherein the insulin is administered orally.

15. The kit according to claim 11, further comprising:
a timing component, said timing component capable of alerting said user to perform an activity, said activity comprising eating.

16. The kit according to claim 11, wherein the bolus and basal rate levels of the delivery of said insulin to said user conform to a pattern of insulin delivery selected from the group consisting of triangular, diamond and irregular.

17. A method for managing blood glucose levels comprising:
monitoring blood glucose levels of a user;
upon detection of a change in said blood glucose levels, initiating a protocol of insulin administration to bring said blood glucose levels to a target level;
adjusting said insulin administration pursuant to said protocol until said blood glucose levels reach said target level; and
upon reaching said target level, discontinuing said insulin administration.

18. The method according to claim 17, wherein bolus and basal rate levels for said insulin administration are customizable to said user and an activity of said user.

19. The method according to claim 17, wherein bolus and basal rate levels for said insulin administration are customizable by said user.

20. The method according to claim 17, wherein bolus and basal rate levels for said protocol of insulin administration to said user conform to a pattern of insulin delivery selected from the group consisting of triangular, diamond and irregular.

21. The method according to claim 17, wherein in said step of adjusting said protocol is modified at least once.
